(19) United States
(12) Patent Application Publication
(10) Pub. No.: US 2016/0331269 A1
(43) Pub. Date: Nov. 17, 2016

(54) FIELD COIL UNIT AND POSITION DETECTION SYSTEM

(71) Applicant: FIAGON AG MEDICAL TECHNOLOGIES, Hennigsdorf (DE)

(72) Inventors: Timo Kruger, Berlin (DE); Dirk Mucha, Berlin (DE)

(73) Assignee: Fiagon AG Medical Technologies, Hennigsdorf (DE)

(21) Appl. No.: 15/029,649

(22) PCT Filed: Oct. 16, 2014
(86) PCT No.: PCT/EP2014/072282

§ 371 Date: Jun. 9, 2016

(30) Foreign Application Priority Data
Oct. 16, 2013 (DE) 10 2013 221 026.4

(51) Int. Cl. A61B 5/06; A61B 90/14; A61B 90/00; A61B 34/20

(52) U.S. Cl. CPC A61B 5/062; A61B 34/20; A61B 90/14; A61B 90/39; A61B 2034/2051; A61B 2034/2072; A61B 2090/3966; A61B 2034/2055

(57) ABSTRACT

The invention relates to a mobile field coil unit for a medical system for detecting the position of a patient, medical equipment and/or prostheses in an operating area, wherein the field coil unit is designed to emit or receive an alternating electromagnetic field. The field coil unit can be arranged movably on the patient or in an area next to the patient for operation as intended, wherein the field coil unit has an outer contour on at least one placement side, which outer contour is designed to surround an object or a part of a patient laterally in order to prevent lateral sliding between the field coil unit and the object or part of the patient. Preferably, the field coil unit is a field generator.

The invention relates to a mobile field coil unit for a medical system for detecting the position of a patient, medical apparatuses and/or prostheses in an operating area, and to such a medical system comprising a field coil unit according to the invention.

In invasive surgical interventions, surgical instruments are moved during an operation by a surgeon in an operating area in the body of a patient. Operating area denotes the space within the patient which is potentially or effectively impaired by the surgical instruments used during the operation. The operation includes introducing surgical instruments into the patient's body and withdrawing them from the patient's body and moving the surgical instruments in the patient's body and using the surgical instruments in a precisely predetermined intervention area arranged in the operating area. Intervention area denotes the part in the operating area which is to be processed by the surgeon. This can involve e.g. tissue to be removed or vessels to be sealed. Accordingly, an operating area can comprise a multiplicity of intervention areas. Alongside the intervention areas, the operating area contains a multiplicity of sensitive structures which are to be protected against damage by the surgical instruments. The sensitive structures include e.g. vessels, organs, nerves, muscles, ligaments, tendons and other, generally intact tissue that is intended to be maintained in order to limit, the effects of the operation to a necessary minimum, since any further impairment of the patient's body during the operation can increase the health risk for the patient and adversely influence the result of the operation.

When invasive surgical interventions are carried out, therefore, use is regularly made of medical systems for detecting the position of a patient, medical apparatuses and/or prostheses in an operating area, also referred to as position detection systems. Position detection systems support the surgeon in the navigation of surgical instruments in the operating area. Known position detection systems support the surgeon particularly when navigating the surgical instrument into the intervention area in the patient's body, when navigating the surgical instrument while carrying out the operative measure, and when navigating the surgical instrument out of the patient's body. The result and the efficiency of the operation to be carried out can be improved thereby. Furthermore, a precise position detection of the surgical instrument can reduce the risk of an unintentional impairment or damage of surrounding tissue or potentially endangered neural pathways in the operating area.

Position detection systems usually detect the co-ordinate transformation between the patient and at least one medical or surgical instrument during the operation. Position information of a multiplicity of different surgical instruments can be determined in many position detection systems. The position information detected is usually visualized together with pre-operatively obtained planning data and/or intraoperatively obtained image data on a monitor. For this purpose, sensors or localizers are arranged at determinable locations of the patient and of the surgical instruments, and their position information in the operating area is determinable by an evaluation unit of the position detection system.

It is known, for example, to provide different medical instruments such as, for example, pointing instruments, aspirators, forceps, needles, scalpels, electrotomes, cauteries and the like, with localizers for determining position information for such a position detection system and to register the respective medical instrument in the position detection system. Registration involves measuring the position of a reference point—usually the working point of the instrument tip—relative to the localizer arranged on the medical instrument and communicating it to the position detection system. Consequently, the position of the reference point and alignment of the medical instrument are known in the position detection system and can be represented as image data on the monitor together with the preoperative and/or intraoperative image data present.

Such position detection systems can comprise for example optical, ultrasound-aided or electromagnetic localizers. Electromagnetic position detection systems are known, for example, which have a field coil unit which is operated as a field generator and which is arranged alongside the patient and generates a generally alternating electromagnetic field in the operating area. Sensor coils are arranged as localizer on a surgical instrument to be navigated in the operating area. The electromagnetic field induces characteristic electric currents in said sensor coils depending on the alignment of the respective sensor coil relative to the electromagnetic field. An evaluation unit—also referred to hereinafter as position detection unit—measures the induced currents and thus determines the position of the sensor coils and thus the position of a surgical instrument equipped with the sensor coils in the operating area.

For exactly determining the position of the surgical instruments in the operating area, it is generally necessary to determine the position of the operating area with respect to the position detection system. This process is referred to as patient registration. In known systems and methods for registering patients in position detection systems, a patient localizer is arranged and usually fixed at a precisely defined location on the surface of the patient's body. The patient localizer is often fixed to the patient by means of a holding device comprising a fixing band having a hook and loop closure. Afterward, the position detection unit determines the position of the patient localizer in the position detection system and determines therefrom the exact position of the patient in the position detection system.

Accordingly, the object of the present invention is to provide a field coil unit for a system for detecting the position of a patient, medical apparatuses and/or prostheses in an operating area, and such a system, having extended possibilities for use.

According to the invention, the object is achieved by means of a mobile field coil unit for a medical system for detecting the position of a patient, medical apparatuses (such as e.g. surgical or other medical instruments) and/or prostheses in an operating area, wherein the field coil unit is designed to emit or to receive an alternating electromagnetic field and is arrangeable moveably on the patient or in a region alongside the patient for operation as intended.

Furthermore, the object is achieved by means of a medical system comprising a field coil unit according to the invention which is to be operated as a field generator, said medical system comprising a patient localizer for detecting the position of the patient relative to the field coil unit to be operated as a field generator and an evaluation unit for determining the position of the field coil unit to be operated as a field generator to the patient localizer. The patient localizer has a field coil unit to be operated as a field sensor and having at least one coil in which a measurable current can be induced depending on the position of the coil in the operating area by the alternating electromagnetic field generated by the field coil unit to be operated as a field generator. The position of the field coil unit to be operated as a field generator relative to the patient localizer having a field coil unit to be operated as a field sensor is determinable by means of the intensity of the current induced in the coil. For navigation with such a system, a surgical instrument to be navigated in an operating area can have as localizer a field sensor having one or a plurality of sensor coils, wherein a voltage induced in a respective sensor coil and/or a current resulting therefrom are/is measured,

Furthermore, the object is achieved by means of a medical system comprising a field coil unit according to the invention which is to be operated as a field sensor, said medical system comprising a patient localizer for detecting the position of the patient relative to the field coil unit to be operated as a field sensor and an evaluation unit for determining the position of the field coil unit to be operated as a field sensor to the patient localizer. The patient localizer has a field coil unit to be operated as a field generator and having at least one coil. The field coil unit to be operated as a field generator generates an alternating electromagnetic field which, depending on the position of the field coil unit to be operated as a field generator in the operating area can induce a measurable current in the field coil unit operated as a field sensor. The position of the field coil unit to be operated as a field generator relative to the patient localizer having a field coil unit to be operated as a field generator is determinable by means of the intensity of the current induced in the coil. For navigation with such a system, a surgical instrument to be navigated in an operating area can have one or a plurality of coils, to which a voltage is applied or is to be applied, such that the coil or coils of the instrument emit(s) an alternating electromagnetic field which can be detected by the field coil unit operated or two be operated as a field sensor.

The field coil unit can be a field generator. In particular, the field coil unit can be designed as a field generator and/or operated as such. In order to operate the field coil unit as a field generator, a voltage, in particular AC voltage, can be applied thereto, such that the field coil unit emits an alternating electromagnetic field.

Alternatively or additionally, the field coil unit can be a field sensor. In particular, the field coil unit can be designed as a field sensor and/or operated as such. The field coil unit can be a sensor coil or comprise such a sensor coil. In order to operate the field coil unit as a field sensor or sensor coil, a voltage induced in the field coil unit and/or a resulting current are/is measured.

In principle, one and the same field coil unit and/or one and the same coil can be operated or be intended to be operated as a field generator and as a field sensor, in particular simultaneously. On one and the same field coil unit and/or one and the same coil, simultaneously a coil impedance and/or an externally induced voltage can be measured or be intended to be measured, and also a voltage generating an alternating electromagnetic field can be applied or intended to be applied thereto. The field coil unit can comprise one, two, three or a higher number of coils. The field coil unit designed as a field generator preferably comprises six coils. The field coil unit designed as a field sensor preferably comprises one, two, or three coils.

The patient localizer can comprise a field coil unit. Preferably, the patient localizer comprises a field sensor or sensor coil as field coil unit. Alternatively, or additionally, the patient localizer can comprises a field generator as field coil unit.

The field coil unit designed as a field generator also generates the electromagnetic field which can be used to detect the position and alignment (that is to say the location) of a medical instrument with corresponding sensors or localizers.

The inventor has recognized that the field coil unit in conventional position detection systems is arranged in a positionally fixed manner, often in a head restraint of the patient, and thus serves as a reference point of the position detection system. Particularly in operations on the spinal column in which computed tomography image data are created intraoperatively, this can result in the creation of the image data being impeded by the field coil unit, such that the field coil unit has to be arranged anew on the patient. However, this has the result that the reference point of the position detection system may be altered and renewed registration of the patient and of the medical instruments may thus be necessary. As a result, the operation is delayed unnecessarily and there is an additional burden on the patient. The field coil unit has on at least one placement side an outer contour designed in such a way that a field coil unit placed on a surface contour of an object or of a patient is prevented from laterally slipping away from the object or from the patient in a positively locking manner by the surface contour of the object or of the patient. Such a field coil unit can be placed loosely on an object or a patient for use as intended, without the need for further securing measures for holding the field coil unit at its position. Accordingly, the position of the field coil unit can easily be altered manually such that possible obstruction of individual operation or intraoperative diagnosis steps by the field coil unit can be prevented in a simple manner during the operation.

Preferably, at least the placement side comprises an elastic outer layer. The outer contour of the elastic outer layer is thus adaptable to the surface contour of the object or of the patient. In this way, the field coil unit is further secured against slipping away from the object or the patient. Furthermore, the object or the patient is impaired to a lesser extent, owing to the flexibility of the outer layer, particularly upon placement of the field coil unit

Preferably, the outer contour of the placement side of the field coil unit is designed substantially as a central trough and/or leadthrough. Such a field coil unit can be placed and positioned for example on the back of the head of a patient lying prone. Furthermore, the field coil unit can be gripped and transported better by virtue of such a form. Preferably, the field coil unit comprises a central leadthrough having an internal diameter having a magnitude at least such that an operative intervention with a catheter can be performed through the leadthrough when the field coil unit is arranged at an operating area on the patient's body.

In a further preferred embodiment variant, the field coil unit comprises a reference receptacle or a referenced stop as a reference point for an instrument. Such a field coil unit allows a simple positional adjustment of a medical instrument with corresponding sensors or localizers, by the instrument being inserted into the referenced receptacle or brought to the referenced stop, because then the actual relative position of instrument and field coil unit with respect to one another is unambiguously known and the signals detected by the sensor or localizers can be correspondingly assigned to this known position.

In a particularly preferred embodiment of the invention, the field coil unit is designed substantially in a ring-shaped fashion, in a toroidal fashion or in a tub-shaped fashion. This has the advantage that the field coil unit can have a very compact design. Furthermore, the field coil unit is ergonomically shaped and presents no corners for possible collisions with persons, medical apparatuses or other objects. A design having the geometry of a tub with a central opening or of the torus bent in the side view additionally offers the abovementioned desirable views that are different depending on the perspective.

Preferably, the field coil unit comprises a fixing device for reversibly fixing the field coil unit to the patient and/or an object. As a result, the position of the field coil unit can be temporarily fixed in the position detection system. The fixing device can comprise e.g. a clamping device or a band.

Particularly preferably, the fixing device comprises a negative-pressure suction device, such as e.g. a suction cup, in order to releaseably fix the field coil unit to a surface by means of negative pressure.

Preferably the field coil unit has a sheathing which encloses the field coil unit substantially in a watertight fashion. Consequently, the field coil unit is protected against external influences, in particular liquids, and can easily be cleaned or disinfected.

With further preference, the sheathing comprises a synthetic polymer, such as silicone, for example, which is particularly suitable as sheathing for the field coil unit owing to its high flexibility or elasticity and water-repellant property. It is furthermore advantageous if the sheathing is molded around the field coil unit or the latter is laminated into the sheathing. Such a sheathing can be produced easily and has particularly good durability and affords good protection of the sheathed components of the field coil unit against environmental influences.

In one advantageous configuration of the invention, the field coil unit has marker points for linking to an optical position detection system. An optical position detection system has the advantage over an electromagnetic position detection system that it is not necessary to generate a further alternating electromagnetic field in order to determine the position of the field coil unit in the position detection system. Consequently, the alternating electromagnetic field generated or received by the field coil unit is not disturbed. An optical position detection system can be used successfully if the field coil unit is arranged as far as possible such that the marker points can be detected by the optical position detection system.

In order to be able to unambiguously detect the position and alignment of the field coil unit in a fluoroscopically recorded image (e.g. x-ray image or CT) the field coil unit preferably has a corresponding, e.g. non-rotationally symmetrical, geometry of its outer form, such that it has specifically different views when observed from respectively different perspectives. Additionally or alternatively, the field coil unit can also comprise fluoroscopically detectable marker points or a corresponding control point network, such that its position in fluoroscopically recorded images is unambiguously determinable.

The medical system according to the invention preferably comprises a patient localizer which is fixable to the patient releaseably. The special feature here is that, in contrast to conventional position detection systems, the patient localizer rather than the field generator is the reference point of the position detection system. This is possible by virtue of the fact that the patient localizer is fixable to the patient in a positionally fixed manner. An alteration of the position of the field generator can easily be determined by the evaluation unit by way of the alteration of the currents induced in the patient localizer. Since the position of the field generator is thus determinable at any time the positions of medical instruments which are arranged in the operating area and are registered in the position detection system are also determinable by the evaluation unit, at any time.

Alternatively, a further position detection sensor could be used as a reference point for the position detection system. For this purpose, the position detection sensor is to be arranged in a positionally fixed manner in the region of the position detection system.

Preferably, the patient localizer has a sheathing which encloses the patient localizer substantially in a watertight fashion. Consequently, the patient localizer is protected against external influences, in particular liquids, and can easily be cleaned or disinfected. This is particularly of importance since the patient localizer is arranged directly on the patient and there is thus a particularly high risk of infection for the patient.

Preferably, the medical system comprises at least one instrument sensor for determining the position in a coordinate system of the position detection system of a medical instrument registered in the position detection system and arranged in the operating area. The instrument sensor is arranged directly on the medical instrument.

The medical system likewise preferably comprises at least one display unit for displaying image data of the patient registered in the position detection system and/or of the medical instruments or prostheses registered in the position detection system. The image data are representable by the display unit alongside one another and/or in a superimposed manner.

Subject matter which is also independently protectable relates to a skull clamp, in particular for patient fixing in the context of neurosurgery. A field coil unit is arranged on the skull clamp. Preferably, the skull clamp has a horseshoe-shaped section for accommodating a skull. With further preference, the field coil unit is designed in a horseshoe fashion. The field coil unit can be arranged congruently with a section of the skull clamp. Preferably, the field coil unit is designed in a horseshoe-shaped fashion and is arranged substantially congruently with the horseshoe-shaped section of the skull clamp. Preferably, the field coil unit is a field generator with further preference having exactly three coils.

The field coil unit designed in a horseshoe-shaped fashion can be fixed to a section of the skull clamp by positively locking engagement, for example by a sleeve. Alternatively or additionally, the field coil unit designed in a horseshoe-shaped fashion can be fixed to a section of the skull clamp by a fastening screw or clamp.

The invention will be explained in greater detail below on the basis of an exemplary embodiment with reference to a drawing, in which:

FIG. 1 shows a plan view of one embodiment of a field coil unit according to the invention designed as a field generator;

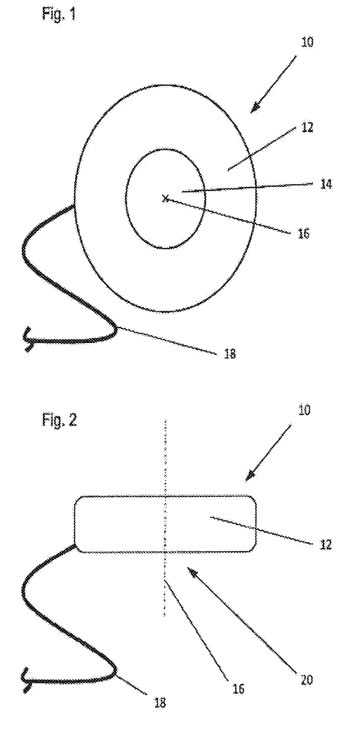

FIG. 2 shows a side view of the field coil unit according to the invention from FIG. 1;

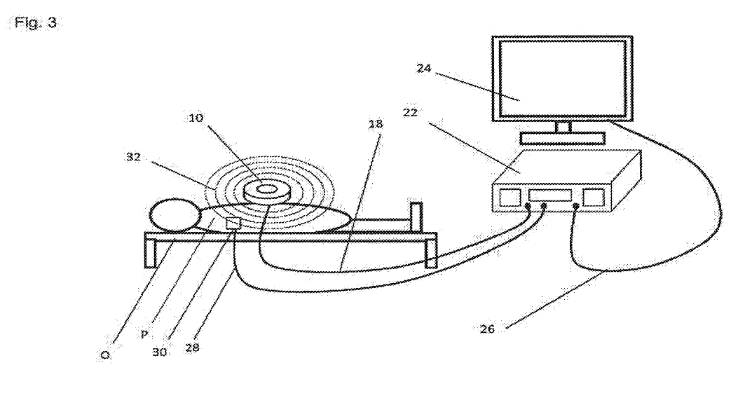

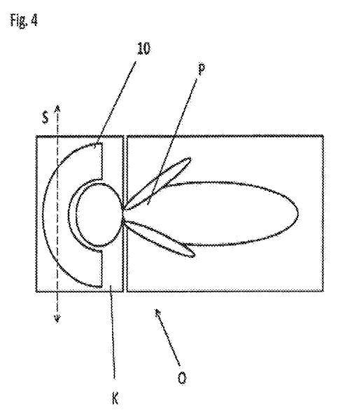

FIG. 3 shows a schematic illustration of a medical system according to the invention for detecting the position of a patient, medical apparatuses and/or prostheses in an operating area;

FIG. 4 shows a schematic illustration of a field coil unit according to the invention designed in a horseshoe-shaped fashion; and

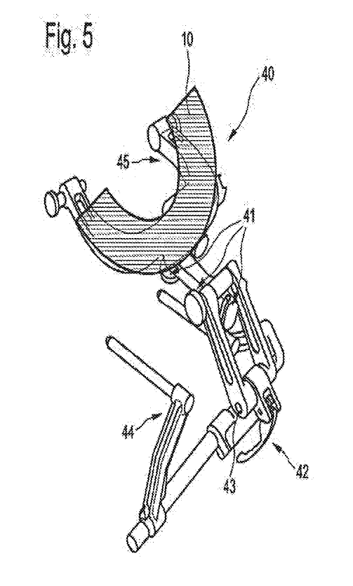

FIG. 5 shows a schematic illustration of skull clamp according to the invention.

The embodiment of the field coil unit 10 according to the invention designed as a field generator, as illustrated in FIG. 1 and FIG. 2, comprises a ring-shaped main body 12 having a top side and an underside and a generator axis 16 extending between the top side and the underside. A leadthrough 14 open on both sides extends from the top side to the underside. The leadthrough 14 has a circular cross-sectional area, the mid point of which is arranged on the generator axis 16. Furthermore, the field coil unit 10 comprises a generator cable 18 for power supply and for control of the field coil unit 10. An alternating electromagnetic field can be generated by the field coil unit 10 according to the invention designed as a field generator.

As is clearly discernable from FIG. 2, the edges of the field coil unit 10 are rounded. Furthermore, the field coil unit 10 is completely surrounded by a sheathing, not discernable in more specific detail in the figures. The sheathing is composed of a flexible, elastic and water-resistant material and preferably has a slip-inhibiting surface. The sheathing thus protects the field coil unit 10 against impacts and the penetration of liquids. The field coil unit 10 has a sufficient strength in order that it is not permanently deformed under intended use conditions. In alternative embodiments, the strength of the first coil unit 10 is not sufficient, and so the sheathing must have a corresponding strength in order to protect the field coil unit 10 against deformations.

One side of the field coil unit 10 is designed as an underside 20 and serves, as shown schematically in FIG. 3, as a placement side of the field coil unit 10 on a patient P or an object such as e.g. an operating table O. In specific embodiments, the underside 20 can have a special coating and/or structure in order to increase e.g. the surface friction coefficient and thus the slip resistance.

FIG. 3 shows, in a schematic illustration, one embodiment of the medical system according to the invention for detecting the position of the patient P, medical apparatuses and/or prostheses in an operating area. The patient P lies supine on the operating table O. A patient localizer 30 is arranged in a positionally fixed manner on the patient P. The patient localizer 30 is releaseably secured to the patient P by fixing means (not depicted), such that the patient localizer 30 cannot alter, or can only minimally alter, its position relative to the patient P during the operation. Therefore, it is particularly expedient for the patient localizer 30 of a medical system according to the invention to be arranged on a body part of the patient P at which the surgical intervention substantially takes place. In the example illustrated, the patient localizer 30 is fixed to the torso of the patient P.

The patient localizer comprises sensor coils (not illustrated) and is connected to an evaluation unit 22 via a patient cable 28. The evaluation unit 22, via the generator cable 18, controls the field coil unit 10 designed as a field generator and thus the generation of the alternating electromagnetic field 32 by the field coil unit 10 designed as a field generator. In the example shown, the field coil unit 10 designed as a field generator is placed by the underside 20 loosely on the stomach of the patient P. Owing to the constitution of the underside 20 and/or the shape of the field coil unit 10 designed as a field generator, the field coil unit 10 designed as a field generator remains on the patient P during the operation, without sliding away laterally, provided that sliding away is not initiated by corresponding external influences, such as e.g. vibrations or shaking.

During intended use of the position detection system, the field coil unit 10 designed as a field generator generates an alternating electromagnetic field 32, illustrated schematically in FIG. 3. The alternating electromagnetic field induces currents in the sensor coils of the patient localizer 30, which currents are communicated to the evaluation unit 22 via the patient cable 28. The intensity of the induced currents is dependent on the position of the individual coils in the alternating electromagnetic field 32. Accordingly, the relative position between the patient localizer 30 and the field coil unit 10 designed as a field generator can thus be determined by the evaluation unit 22. Since the position of the patient localizer 30 within the position detection system is known, the exact position of the field coil unit 10 designed as a field generator is thus determinable by the evaluation unit 22 even if the position of the field coil unit 10 designed as a field generator is altered—e.g. owing to a possible collision with a computed tomography apparatus during the intraoperative creation of computed tomography images of the patient P.

A display unit 24 is connected to the evaluation unit via a display cable 26. Preoperatively and intraoperatively obtained image data of the patient P and image data of medical instruments are representable positionally faithfully alongside one another or in a superimposed manner on the display unit 24. The navigation of the medical instruments in the operating area is thus made considerably easier for the surgeon.

FIG. 4 shows a plan view of a patient P lying supine on an operating table O. The head of the patient P lies on a head restraint K of the operating table O. A mobile field coil unit 10 in FIG. 4 is designed in a horseshoe-shaped fashion and arranged moveably in a region alongside the patient P by being placed onto the head restraint K. In the present case, the mobile field coil unit 10 has on its placement side a horseshoe-shaped outer contour designed to laterally surround the head of the patient P. This prevents sliding away between the field coil unit 10 and the head of the patient P in a lateral direction S.

FIG. 5 shows a skull clamp 40, which can be secured to an operating table (not illustrated) by means of a table adapter 44. The skull clamp 40 is connected to the table adapter 44 moveably, in the present case pivotably, via an axial joint 43. The axial joint 43 can be fixed via a fixing lever 42. The skull clamp 40 has relative to the axial joint 43 further degrees of freedom which can be locked by one or a plurality of fixing knobs 41. The skull clamp 40 has a horseshoe-shaped section 45 for accommodating a skull, on which the field coil unit 10 designed in a horseshoe-shaped fashion in the present case is arranged. As is evident from FIG. 5 the field coil unit 10 designed in a horseshoe-shaped fashion is arranged substantially congruently with horseshoe-shaped section 45 of the skull clamp 40.

The horseshoe-shaped section 45 of the skull clamp 40 has an H-shaped cross section via which the field coil unit 10 designed in a horseshoe-shaped fashion is fixed to the horseshoe-shaped section 45 of the skull clamp by positively locking engagement. The field coil unit 10 designed in a horseshoe-shaped fashion is arranged on the skull clamp 40 in a balanced manner in such a way that even in the case of a width adjustment of the skull clamp 40 the field coil unit 10 designed in a horseshoe-shaped fashion is arranged substantially congruently with the horseshoe-shaped section 45 of the skull clamp 40.

The use of a field coil unit according to the invention by way of example in a method for incorporating image data obtained by computed tomography and/or x-ray technology into a system for operation planning and/or for intraoperative navigation will be explained in greater detail below. In this case, it is preferred for the field coil unit to be designed in a ring-shaped fashion. The method comprises the following steps:
recording tomographic image data or image data obtained by x-ray technology from at least one defined body region of the patient by means of at least one first recording apparatus suitable for this purpose, wherein the field coil unit is arranged as a first reference body with at least one surface on the patient and is concomitantly recorded by the first recording apparatus;
comparing the recorded image data representing the first reference body with known geometrical data of the first reference body for obtaining distortion information;
eliminating distortion from the recorded image data by means of a computing unit on the basis of the distortion information for obtaining image data from which distortion has been eliminated;
superimposing the image data from which distortion has been eliminated with further image data of the same body region of the patient for obtaining superimposed image data; and
representing the superimposed image data on display.

The method includes the concept of preoperatively producing a tomogram obtained by computed tomography and intraoperatively producing x-ray images (fluoroscopic images) using a C-arm. By virtue of the body having known geometry, distortion can be eliminated from the intraoperatively recorded x-ray images (fluoroscopic images) and the latter can thus be compared with x-ray images generated virtually, from the preoperatively recorded, for example by a computed tomography apparatus. This has the advantage that a physician in the course of operation planning and when carrying out the operation can be oriented by x-ray images, to be precise both preoperatively recorded and intraoperatively recorded x-ray images. Instead of image data obtained by x-ray technology, image data can also be recorded by some other imaging method, e.g. an ultrasound-based method. Likewise, the tomographic; image data can be obtained by means of a computed tomography apparatus, a magnetic resonance imaging apparatus, an ultrasound tomography apparatus or the like.

The field coil unit is preferably a field generator.

Preferably, the geometrical data of the first reference body are present as image data which image the actual geometry of the fist reference body without distortion.

Preferably, the further image data of the same body region of the patient comprise the geometrical data of the first reference body as image data.

Furthermore, it is preferred if the first reference body, during the recording of the further image data, has the same position with respect to the recording apparatus and/or patient as during the recording of the tomographic image data, in order not to have to take account of different positions when eliminating distortion from the image data.

Moreover, it is advantageous if the first reference body, during the recording of the further image data, is at the same distance from the recording apparatus as during the recording of the tomographic image data, such that, if possible, different distortions do not occur.

It is also advantageous if the first reference body during the creation of the tomographic image data, is aligned in such a way that the greatest possible part of the surface of the first reference body faces the recording apparatus, in order in this way to be able to record image data that are as significant as possible from the reference body.

Preferably, the first reference body is arranged at a body location of the patient, such that the body location and the first reference body can be detected with a recording that is as detailed as possible.

The further image data of the same body region of the patient can be created by a second recording apparatus, preferably an x-ray apparatus, and comprise an x-ray image or fluoroscopic images.

The first reference body is preferably ring-shaped or circular and/or has ring-shaped and/or circular regions and/or elements.

Preferably, the further image data are recorded intraoperatively and are thus extremely up to date.

The display preferably comprises a monitor.

The defined body region of the patient preferably comprises the spinal column, that is to say that the method is advantageous particularly for supporting examinations or treatments in the region of the spinal column.

At least some other tomographic image data can be recorded before an examination or operation, e.g. in a tomography apparatus, for instance a magnetic resonance imaging apparatus, which would otherwise hinder the examination or operation.

Particularly preferably, a second or further reference body is arranged at an operating site in the patient's body, wherein the operating site is arranged in the defined body region of the patient. The first reference body and the second reference body are registered in a navigation system for planning and carrying out operations, wherein position data of the first reference body and of the second reference body relative to a reference point are determined by the navigation system. For registration purposes it is advantageous if the first reference body and the second reference body have localizers, such as e.g. sensor coils. The use of sensor coils in such navigation systems is known in principle. By means of the determined position data of the first reference body and of the second reference body, the tomographic image data and further image data can be represented in a superimposed manner on the display in the navigation system. This enables an accurate navigation with the aid of intraoperatively obtained x-ray images from which distortion has been eliminated. The accuracy of the superimposition of the image data is optimized by the registration of the first reference body and of the second reference body in the navigation system.

The use of two reference bodies in combination with one another, of which a first is the body having known geometry and the second is a smaller local localizer at the operating site, allows spatial information to be obtained intraoperatively and to be inserted into x-ray images generated intraoperatively.

It is also possible for two of the reference bodies to be bodies having known geometry. This makes it possible to obtain spatial information for example from fluoroscopic images obtained by x-ray technology in part, particularly if a plurality of two-dimensional images without depth information (e.g. images obtained by x-ray technology) were recorded from different perspectives and related to one another.

Additionally or alternatively, at least some of the tomographic image data or image data obtained by x-ray technology can be recorded during an operation (intraoperative) and are thus in each case entirely up to date. In particular, it is appropriate to record image data intraoperatively by means of a compact x-ray apparatus such as e.g. a C-arm. Often such intraoperatively recorded image data are fluoroscopic images without depth information, and contain no tomograms. Spatial information can then nevertheless be obtained with the aid of one or a plurality of reference bodies of known geometry. This last holds true primarily—but not exclusively—when for example preoperatively obtained image data having spatial information, e.g. tomograms such as tomograms obtained by computed tomography or magnetic resonance imaging tomograms, are also present which are linked intraoperatively with intraoperatively obtained image data.

Furthermore, the field coil unit according to the invention can be used in an alternative medical system for operation planning and/or intraoperative navigation. The alternative medical system comprises at least one computing unit, at least one first recording unit for recording tomographic image data of a body region of a patient, at least one second recording unit for recording further image data of the same body region of the patient, and a first reference body, wherein the medical system is designed for carrying out a method of the type described above.

### LIST OF REFERENCE SIGNS

10 Field coil unit

12 Main body

14 Leadthrough

16 Generator axis

18 Generator cable

20 Underside

22 Evaluation unit

24 Display unit

26 Display cable

28 Patient cable

30 Patient localizer

32 Electromagnetic field

40 Skull clamp

41 Fixing knob

42 Fixing lever

43 Axial joint

44 Table adapter

45 Horseshoe-shaped section of the skull clamp

K Head restraint of the operating table

O Operating table

P Patient

1. A mobile field coil unit for a medical system for detecting the position of a patient, medical apparatuses and/or prostheses in an operating area, wherein the field coil unit is designed to emit or to receive an alternating electromagnetic field,
wherein the field coil unit is arrangeable moveably on the patient or in a region alongside the patient for operation as intended, wherein the field coil unit has on at least one placement side an outer contour designed so as to laterally surround an object or a part of a patient, in order to prevent lateral sliding away between the field coil unit and the object or part of the patient.

2. The field coil unit as claimed in claim 1,
wherein at least the placement side has an elastic outer layer, wherein the outer contour of the elastic outer layer is adaptable to the surface contour of the object or of the patient.

3. The field coil unit as claimed in claim 2,
wherein the outer contour of the placement side is designed substantially as a central trough and/or leadthrough for leading through a catheter hose or tube.

4. The field coil unit as claimed in claim 2,
wherein the field coil unit is designed substantially in a ring-shaped fashion, in a toroidal fashion or in a tublike fashion.

5. The field coil unit as claimed in claim 1,
wherein the field coil unit is designed in a horseshoe-shaped fashion.

6. The field coil unit as claimed in claim 1,
wherein the field coil unit comprises a fixing device for reversibly fixing the field coil unit to the patient and/or an object.

7. The field coil unit as claimed in claim 6,
wherein the fixing device comprises a negative-pressure suction device.

8. The field coil unit as claimed in claim 1,
wherein the field coil unit has a sheathing which encloses the field coil unit substantially in a watertight fashion.

9. The field coil unit as claimed in claim 8,
wherein the sheathing comprises a synthetic polymer, and in that the sheathing is molded around the field coil unit or the latter is laminated into the sheathing.

10. The field coil unit as claimed in claim 1,
wherein the field coil unit has marker points for linking to an optical or fluoroscopic position detection system.

11. The field coil unit as claimed in claim 1,
wherein the field coil unit is a field generator.

12. A skull clamp, in particular for patient fixing in the context of a neurosurgical treatment, comprising a field coil unit as claimed in claim 1, wherein the field coil unit is arranged on the skull clamp, wherein the field coil unit is designed in a horseshoe-shaped fashion and is arranged substantially congruently with a section of the skull clamp.

13. A medical system for detecting the position of a patient, medical apparatuses and/or prostheses in an operating area in the region of a body of a patient, comprising:
a field generator for emitting an electromagnetic field;
a patient localizer for detecting the position of the patient relative to the field generator, wherein the patient localizer has at least one coil, wherein a measurable current can be induced in the coil depending on the position of the coil in the operating area by means of the alternating electromagnetic field generated by the field generator, wherein the position of the field generator relative to the patient localizer can be determined by means of the intensity of the current induced in the coil; and
an evaluation unit for determining the position of the field generator relative to the patient localizer;
wherein the field generator has the features of a field coil unit of claim 1.

14. The system as claimed in claim 13,
wherein the patient localizer is fixable to the patient releaseably.

15. The system as claimed in claim 13,
wherein the patient localizer has a sheathing which encloses the patient localizer substantially in a watertight fashion.

16. The system as claimed in claim 15,
wherein the sheathing comprises a synthetic polymer, and in that the sheathing is molded around the patient localizer or the latter is laminated into the sheathing.

17. The system as claimed in claim 13, comprising:
at least one instrument sensor for determining the position in a coordinate system of the position detection system of a medical instrument registered in the position detection system and arranged in the operating area, wherein the instrument sensor is arranged directly on the medical instrument.

18. The system as claimed in claim 13, comprising
at least one display unit for displaying image data of the patient registered in the position detection system and/or of the medical instruments or prostheses registered in the position detection system.
